(19) United States
(12) Patent Application Publication
(10) Pub. No.: US 2010/0312343 A1
(43) Pub. Date: Dec. 9, 2010

(54) TIP SUPPORT INSERT FOR APPLICATION TO LEFT/RIGHT ARTICULAR PROCESSES TO MINIMIZE ABRASION BETWEEN VERTEBRAE AND TO MAINTAIN PROPER ANGLE/LIFT FOR REDUCING NERVE COMPRESSION

(75) Inventor: Miguel A. Linares, BLOOMFIELD HILLS, MI (US)

Correspondence Address: GIFFORD, KRASS, SPRINKLE,ANDERSON & CITKOWSKI, P.C, PO BOX 7021, TROY, MI, 48007-7021, US

(73) Assignee: LINARES MEDICAL DEVICES, LLC, Auburn Hills, MI (US)

(21) Appl. No.: 12/786,118

(22) Filed: May 24, 2010

Related U.S. Application Data
(60) Provisional application No. 61/184,193, filed on Jun. 4, 2009.

(51) Int. Cl. A61F 2/44

(52) U.S. Cl. 623/17.11

(57) ABSTRACT

An insert for a tip of a spinal process portion of a selected vertebrae in communication with a succeeding vertebrae and for providing proper angle and lift between the succeeding vertebrae and for preventing bone-on-bone contact. An insert body exhibits a pocket shaped recess and is constructed of a plastic or like composite material. The body further exhibits an irregular underside surfaces associated with the recess for promoting internal bone growth and adhesion. A rounded exterior surface further promotes seating support between the selected and projecting spinal process upon which the body mounted, as well as an opposing location associated with a successively located vertebrae.

### CROSS-REFERENCE TO RELATED APPLICATIONS

This Application claims the benefit of U.S. Provisional Application 61/184,193 filed on Jun. 4, 2009.

### FIELD OF THE INVENTION

The present invention discloses a tip support for securing upon a terminating portion of an articular process associated with a spinal vertebrae, including such as a lumbar vertebrae, and in order to prevent rubbing with adjoining vertebrae and further in order to maintain proper angle and lift of the vertebrae to reduce compression around the spinal nerve and associated branching networks.

### BACKGROUND OF THE INVENTION

The prior art is documented with various types of spinal fusion or immobilization technologies intended to restrict movement or interaction between either misaligned or damaged spinal vertebrae. This can in one instance include techniques so extreme as screwing a titanium plate to locations associated with two or more vertebrae and in order to prevent pain or discomfort resulting from undesired interaction between the vertebrae, including the spinal process locations associated with each vertebrae, and/or pinching of the associated spinal nerve column and branches.

### SUMMARY OF THE INVENTION

An insert for a tip of a spinal process portion of a vertebrae in communication with a succeeding vertebrae and for providing proper angle and lift between the succeeding vertebrae and for preventing bone-on-bone contact. A plastic or composite insert body exhibits a generally pocket shaped recess. The body exhibits irregular underside surfaces associated with the recess interior for promoting internal bone growth and adhesion with the contacting marrow. A rounded exterior surface further promotes seating support between the selected and projecting spinal process upon which the body mounted, as well as an opposing location associated with a successively located vertebrae.

### BRIEF DESCRIPTION OF THE DRAWINGS

Reference will now be made to the attached drawings, when read in combination with the following detailed description, wherein like reference numerals refer to like parts throughout the several views, and in which:

FIG. 1A is a Prior Art rear view of an existing lumbar vertebrae and showing an area of abrasion upon a surface location of an associated process resulting from rubbing between succeeding vertebrae;

FIG. 1B is a Prior Art rotated side view of the lumbar vertebrae shown in FIG. 1A;

FIG. 1C is a Prior Art perspective view of the lumbar vertebrae in FIG. 1A;

FIG. 2A is a Prior Art rear view of a plurality of lumbar vertebrae (selected from L1-L5) arranged in aligned and stacked fashion relative to a spinal column;

FIG. 2B is a Prior Art side view of the lumbar vertebrae shown in FIG. 2A;

FIG. 3A is an illustration of a tip support insert for securing over a selected articular or transverse process associated with a lumbar vertebrae;

FIG. 3B is a cutaway view taken along line 3B-3B of FIG. 3A and showing the configuration of the plastic material of the tip support in formed fashion about a tip portion associated with the selected vertebral processes;

FIG. 4 is an illustration of a tip support illustrated in affixed fashion to a terminating process portion of an existing lumbar vertebrae;

FIG. 5 is a cutaway illustration showing an optional undercut pattern formed within interior surfaces of the tip insert for promoting bone marrow growth and fusion between the surface of the process and the adhering surfaces of the tip insert; and

FIG. 6 is a partial view further representative to that shown in FIG. 5 and illustrating the plastic to bone interface established between the tip insert and the projecting spinal process.

### DETAILED DESCRIPTION OF THE PREFERRED EMBODIMENTS

As will be described with subsequent reference to the several detailed illustrations provided herein, the present invention discloses a tip support for securing upon a terminating portion of an articular process associated with a spinal vertebrae, including such as a lumbar vertebrae. In use, the tip support prevents undesirable rubbing or abrasion which can occur between a surface of a vertebral process and a receiving contact surface (e.g. cavity or pocket) associated with adjoining vertebrae, as well as further in order to maintain proper angle and lift between the vertebrae to reduce compression around the spinal nerve and associated branching networks.

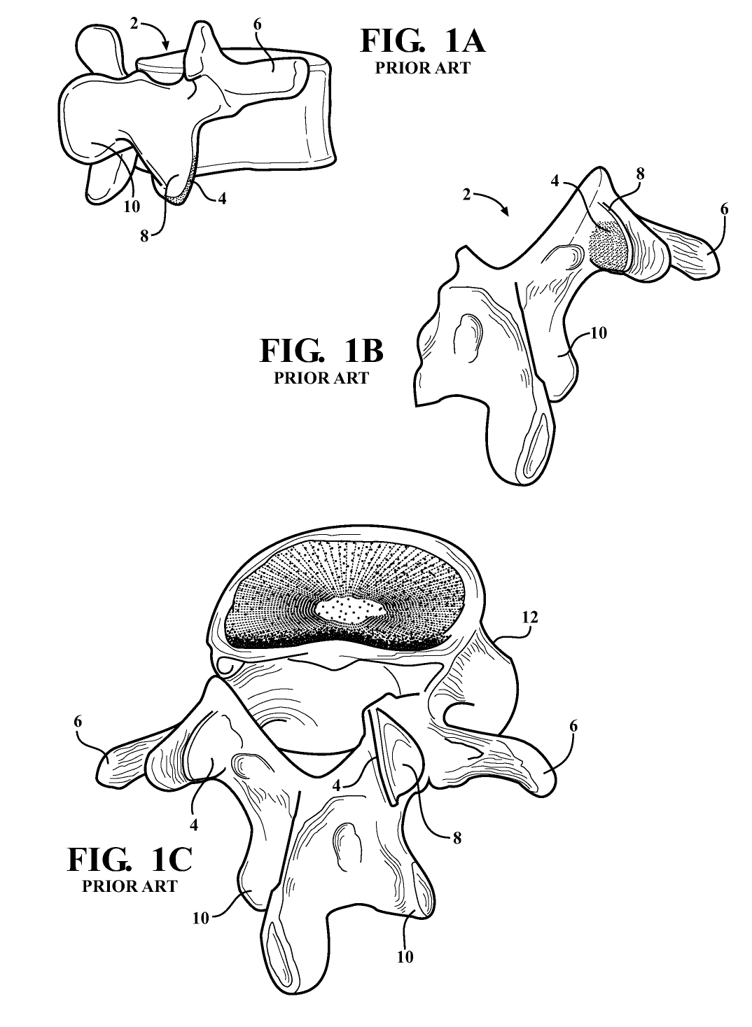

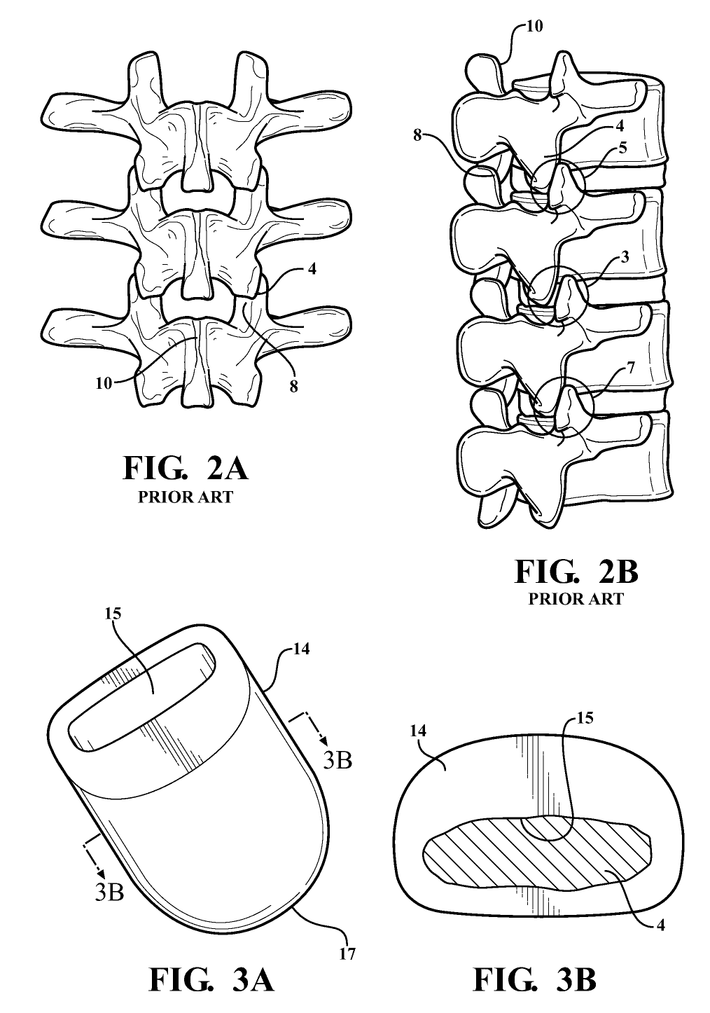

Referring first to the several Prior Art illustrations of FIGS. 1A-1C and FIGS. 2A-2B, shown are typical lumbar vertebrae associated with a human spinal column. As is known, the lumbar portion of the spinal column consists of vertebrae L1-L5, these being positioned in adjoining and extending fashion along a lower third of the spinal column (below the uppermost cervical and middle positioned thoracic vertebrae) as well as above the lower-most positioned Os sacrum and coccyx vertebrae.

FIG. 1A-1C illustrate successive rear, side and perspective views of an existing lumbar vertebrae 2 and showing, such as along each of identical first and second sides, an area of abrasion which is illustrated along a surface associated with at superior articular process 4, this resulting from rubbing between succeeding vertebrae (and more specifically between an end face of the process of the first vertebrae relative to the seating recess or pocket associated with the succeeding vertebrae) as further shown in stacked fashion in each of FIGS. 2A and 2B. In the illustrated example, the abraded area corresponds in location to a region located between a superior articular process (at point of abrasion 4) and reverse face located mammillary articular process 8 (and which establishes seating support to a further process 4 associated with a successive interacting vertebrae).

An anatomical review of the selected lumbar vertebrae 2 also depicts, additional to the superior articular process 4 and mamillary articular process 8, a transverse processes 6, and, further at 10, an extending portion associated with an inferior articular process location. Further shown in FIG. 1C is a main body portion 12 associated with each vertebrae and which, by configuration, is flattened or slightly concave above and below, concave behind, and deeply constricted in front and at the sides.

As best shown in FIGS. 2A and 2B, the seating arrangement established between the superior articular process 4 at the indicated point of abrasion on the selected vertebrae 2 and a corresponding underside of the mamillary process 8 which is particularly susceptible to abrasion and/or misalignment, this also resulting from associated misalignment resulting from inter-movement of the individual vertebrae within the spinal column. The rotated side view of FIG. 2B is especially illustrative of a potential misalignment incident (see depicted area 3 in relation to upper area 5 and lower area 7 established between succeeding lumbar vertebrae)

By way of additional explanation, and of the various processes shown, the superior articular process 4 and inferior articular process 10 are well-defined, projecting respectively upward and downward from the junctions of pedicles and laminae. The facets on the superior processes 4 are concave (see again as best shown by abraded area and as again designated at 4 in FIG. 1C), and look backward; those on the inferior 10 (again FIG. 1C) are convex, and are directed forward and lateral-ward. As is further known, the former (superior 4) are wider apart than the latter (inferior 10), since in the articulated column the inferior articular processes are embraced by the superior processes of the subjacent vertebra.

The transverse processes 6 are long and slender, as well as horizontal in the upper three lumbar vertebrae (L1-L3) and incline a little upward in the lower two (L4-L5). In the upper three vertebrae they arise from the junctions of the pedicles and laminae, but in the lower two they are set farther forward and spring from the pedicles and posterior parts of the vertebral bodies and are further situated in front of the articular processes, instead of behind them as in the thoracic vertebrae.

Of the three tubercles noticed in connection with the transverse processes 6 of the lower thoracic vertebrae, the superior one is connected in the lumbar region with the back part of the superior articular process, and is named the mammillary process. The inferior is situated at the back part of the base of the transverse process, and which is called the accessory process.

Having undertaken a general explanation of the construction of the lumbar vertebrae, a description of the tip support insert of the invention will now be made. It is also further understood that application of the tip support insert is not limited necessarily to use with various process portions associated with a lumbar vertebrae, but can also be modified for use with any extending process portion associated with either the cervical or thoracic vertebrae, and which may further be subject to misalignment and/or abrading contact with succeeding vertebrae in a similar fashion as depicted herein.

The above said, FIG. 3A is an illustration of a tip support insert 14 for securing over a selected articular or transverse process associated with a lumbar vertebrae. The tip support insert 14 as shown exhibits a three dimensional shaped article, such as is constructed of a plastic or composite plastic material and with an interiorly formed pocket defined by continuous inner and cavity defining wall 15 which is configured for being installed in supporting fashion over a terminating tip portion of a spinal process portion (such as again primarily upon a superior articular process for providing an abrading and misalignment resistant pocket with a succeeding and underside seating location associated with such as a mamillary/accessory process portion. In one embodiment, the exterior configuration of the tip insert 14 is such that it exhibits a rounded surface 17 which promotes seating support between the projecting articular process upon which it is mounted, as well as the opposing location of the successively located vertebrae.

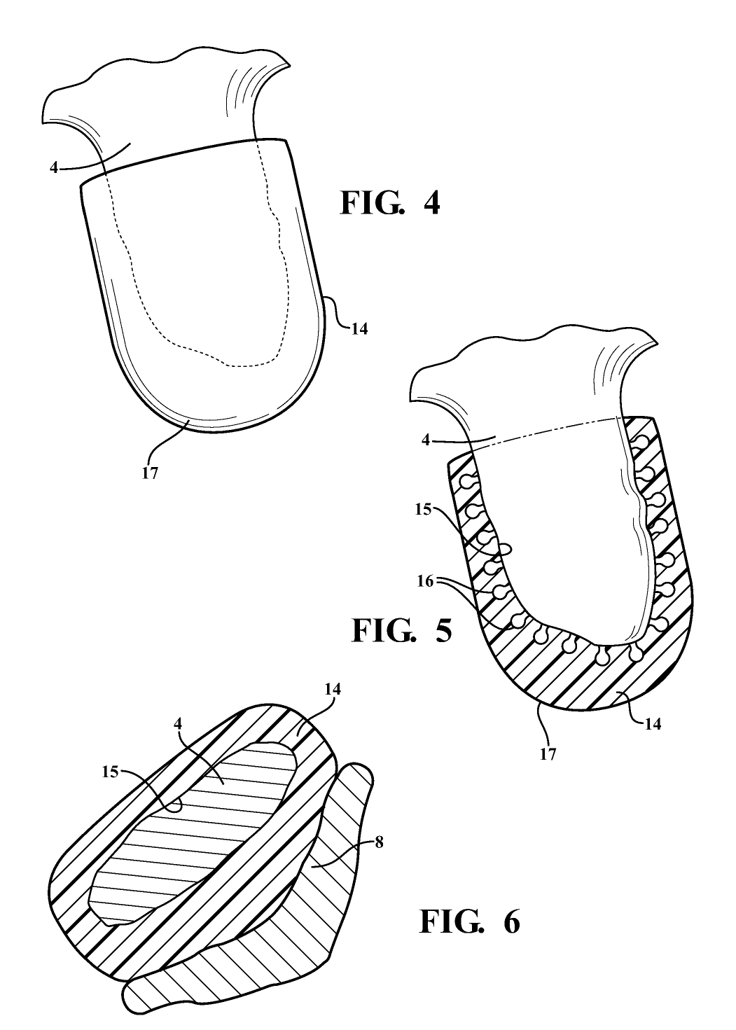

FIG. 3B is a cutaway view taken along line 3B-3B of FIG. 3A and showing the configuration of the plastic material of the tip support 14 in formed fashion about a tip portion associated with the selected vertebral processes (see again terminating finger portion of superior articular process 4). FIG. 4 is an illustration of the tip support 14 fashioned about the terminating process portion (see also in phantom) 4 of the existing lumbar vertebrae.

Further depicted in the cutaway view of FIG. 5 is the manner in which a modification of the insert can exhibit an undercut or irregular surface pattern (see undercut pattern defining locations 16) extending along its interior defining surface (such as again shown at 15) associated with the pocket defined in the insert, and in order to promote fusion with bone growth associated with the vertebrae in order to permanently affix the spacer tip insert 14 in place upon the terminating finger portion of the spinal process 4.

The irregular/undercut surface pattern 16 promotes bone marrow growth and fusion between the surface of the process 4 and the adhering surfaces of the tip insert 14. It is further envisioned and understood that, without limitation, other mechanical fasteners or adhesives can also be employed for permanently affixing the tip insert 14 in place upon the terminating tip portion of any selected bone process not limited to the superior articular process 4 and which operates to establish either or both a desired spacing or inter-vertebral contact surface between a selected process of a first vertebrae and a seating pocket or any other concave or receiving formation or configuration associated with a successive vertebrae.

Finally, and referring to FIG. 6, a partial view is shown which is further representative to that shown in each of FIGS. 3B and 5 and illustrates from a side cutaway profile the cushioning interface established between the interposed plastic sleeve 14 and superior articular process 4 of a first vertebrae and seating/receiving mamillary process 8 of a succeeding vertebrae, this again and in order to prevent bone-to-bone abrading contact between overlapping process portions and such as further resulting from misalignment existing elsewhere in the spinal vertebrae and/or adjoining vertebrae. Reference is also once again made to inter-vertebral contact locations illustrated in FIGS. 2A and 2B, and for which installation of an appropriately configured insert 14 of desired thickness and dimension is useful for both voiding inter-vertebral bone on bone rubbing (see again process locations 4 and 8 associated with succeeding vertebrae), as well as to restore proper angle and/or lift between the vertebrae in order to reduce compression forces around the associated spinal nerve and associated branches.

Having described my invention, other and additional preferred embodiments will become apparent to those skilled in the art to which it pertains, and without deviating from the scope of the appended claims.

I claim:

1. An insert for installation over a tip of a spinal process portion for providing proper angle and lift between succeeding vertebrae and for preventing bone-on-bone contact, said insert comprising a body having a pocket shaped recess.

2. The invention as described in claim 1, said body being constructed of at least one of a plastic or a composite plastic material.

3. The invention as described in claim 1, said body further exhibiting an irregular underside surfaces associated with said recess for promoting bone growth and adhesion.

4. The invention as described in claim 1, said body further comprising a rounded exterior surface which promotes seating support between the projecting articular process upon which it is mounted, as well as the opposing location of the successively located vertebrae.

5. An insert for installation over a tip of a spinal process portion of a first vertebrae in contact with a location of a succeeding vertebrae, comprising:
a three dimensional shaped body exhibiting an interiorly formed pocket defined by continuous inner and cavity defining wall which is applied over a tip of the process portion;
said article providing proper angle and lift between the first vertebrae and the succeeding vertebrae.

6. The invention as described in claim 5, said body being constructed of at least one of a plastic or a composite plastic material.

7. The invention as described in claim 5, said body further exhibiting an irregular underside surfaces associated with said recess for promoting bone growth and adhesion.

8. The invention as described in claim 8, said body further comprising a rounded exterior surface which promotes seating support between the projecting articular process upon which it is mounted, as well as the opposing location of the successively located vertebrae.

9. An insert for installation over a tip of a spinal process portion of a first vertebrae in contact with a location of a succeeding vertebrae, comprising:
a three dimensional shaped composite plastic body exhibiting an interiorly formed pocket defined by continuous inner and cavity defining wall which is applied over a tip of the process portion, an irregular surface exhibited upon said inner cavity defining wall for promoting bone growth and adhesion;
said body further comprising a rounded exterior surface which promotes seating support between the projecting articular process upon which it is mounted, as well as the opposing location of the successively located vertebrae; and
said article providing proper angle and lift and preventing bone on bone contact between the first vertebrae and the succeeding vertebrae.
